(19) United States
(12) Patent Application Publication
(10) Pub. No.: US 2019/0329016 A1
(43) Pub. Date: Oct. 31, 2019

(54) CONNECTION ADAPTER FOR CONNECTING A FLUID LINE OF A DEVICE FOR VACUUM INFUSION

(71) Applicant: Airbus Operations GmbH, Hamburg (DE)

(72) Inventors: Remo HINZ, Hamburg (DE); Marc MAYER, Hamburg (DE)

(21) Appl. No.: 16/395,713

(22) Filed: Apr. 26, 2019

(30) Foreign Application Priority Data
Apr. 27, 2018 (DE) 102018110162.7

(51) Int. Cl. A61M 39/10; A61M 39/02

(52) U.S. Cl. CPC A61M 39/10; A61M 2039/1077; A61M 39/02

(57) ABSTRACT

A connection adapter for conducting resin into an inner fluid line of a device for vacuum infusion has a bearing side and a connection side, wherein the bearing side has two bearing legs for placing on a fluid line which form an interspace which widens outwardly in a direction facing away from the connection side, and wherein the connection side has a hollow-cylindrical portion with a connection opening facing away from the bearing side, and with a connecting opening projecting into the bearing side, and with a fluid connection between the connection opening and the connecting opening.

### CROSS-REFERENCES TO RELATED APPLICATIONS

This application claims the benefit of the German patent application No. 10 2018 110 162.7 filed on Apr. 27, 2018, the entire disclosures of which are incorporated herein by way of reference.

### FIELD OF THE INVENTION

The invention relates to a connection adapter for connecting an inner fluid line of a device for vacuum infusion, and to a system for carrying out a vacuum infusion method.

### BACKGROUND OF THE INVENTION

Various methods are known for the production of components from fiber-reinforced plastics. The choice of a suitable method is often based on the intended application and on a planned number of components to be produced. Relatively large quantities and relatively large component dimensions are often managed by vacuum infusion methods in which fiber semi-finished products are introduced into a mold, are covered by a gas-tight film and then, by applying a negative pressure, are supplied with resin from a resin line situated below the film. In order to supply this inner resin line with a sufficient flow of resin, it is essential to produce a connection between the inner resin line and an outer resin line. Since the inner resin line is customarily placed directly in or on the fiber semi-finished product, the production of the connection can be laborious.

It is known practice to connect hoselines or the like to such inner resin lines, with the result that the hoselines then extend out of the fiber semi-finished product underneath the film until they project from the mold. It is thus possible, depending on the configuration of the fiber semi-finished product, for an impression of the hoseline to subsequently appear on the produced component.

For example, DE 10 013 409 C1 shows a method for producing fiber-reinforced plastic components from dry fiber-composite semi-finished products by means of an injection method in which such hoselines are used to supply the fiber semi-finished products with resin.

### SUMMARY OF THE INVENTION

It can be considered as an object to improve known methods to the effect that the connection of a fluid line to an external fluid source, for example a resin line, or fluid sink, for example a vacuum pump, can be carried out without causing impressions or other points to be corrected on the component to be produced.

What is proposed is a connection adapter for connecting an inner fluid line of a device for vacuum infusion, wherein the connection adapter has a bearing side and a connection side, wherein the bearing side has two bearing legs for placing on a fluid line which form an interspace which widens outwardly in a direction facing away from the connection side, and wherein the connection side has a hollow-cylindrical portion with a connection opening facing away from the bearing side, and a connecting opening projecting into the bearing side, and a fluid connection between the connection opening and the connecting opening.

The connection adapter achieves a number of advantages. On the one hand, it is no longer necessary to lay a separate hoseline underneath a vacuum film, thereby preventing any geometric impairment of a component to be produced. On the other hand, it is possible, virtually at any desired points on the mold, to mount such a connection adapter at desired points of each inner fluid line and, by (pre-)drilling of a hole into the inner line, to connect it thereto.

It should be pointed out that a connection adapter according to the invention can be realized not only for conducting resin from outside into an inner resin line as fluid line. The connection adapter can also be used to suction air from the mold if said adapter is connected to an inner vacuum line as fluid line.

Resin or air is conducted in a simple manner by placing the connection adapter and connecting it to the inner fluid line by (pre-)producing a hole, with the result that resin flows from above through the connection adapter into the inner fluid line, or air can be directly suctioned out of the corresponding fluid lines from above the mold. Therefore, a feed line is arranged nowhere underneath the film.

The connection adapter has a bearing side and a connection side for this function. The bearing side is the side which faces the inner fluid line, i.e., the side which bears on the inner fluid line. The connection side is the side which can be connected, for example, to a hose or another device in order to conduct the resin though the connection adapter into the inner resin line or to conduct the air out of the inner vacuum line. The connection opening opens into an upper side of the connection adapter and can thereby be used for connection from above the film.

The inner fluid line, and in particular the inner resin line, are often arranged on an upper side of the fiber semi-finished product, with the result that they can become apparent through the film and can therefore be very readily provided with one or more connection adapters. For the use of the connection adapter according to the invention, it is therefore recommended for the inner fluid lines always to be arranged on upper sides of the fiber semi-finished product. The bearing side of the connection adapter therefore has two bearing legs which substantially form a V shape. This widens outwardly, i.e., in a direction facing away from the connection side. The connection adapter can therefore be placed in the manner of a saddle on an inner fluid line.

The connection side having the hollow-cylindrical portion arranged thereon thus serves to connect a necessary hoseline to the connection adapter. The hollow-cylindrical extent therefore makes it possible for the hose to be plugged into the connection adapter by a desired plug-in depth in order to produce the desired fluid connection. The connection side should be designed in such a way that the film hugs it harmonically. The hollow-cylindrical portion is therefore preferably rounded off on all edges and is as flat as possible.

Since an inner fluid line can also be connected to a plurality of connection adapters, the achievable volumetric flow of the corresponding fluid in the inner fluid line can additionally be readily influenced. The more connection adapters that are used, the higher the volumetric flow into or out of the fluid line can be.

Overall, the connection adapter can therefore particularly improve the feed of resin into an inner resin line or the introduction of a vacuum into a mold of a resin infusion system without having to rely on complicated line systems which extend underneath the vacuum film over or on the component edges. Moreover, the volumetric flow of resin, which flows into the inner resin line(s), or of air, which flows outwardly from inner vacuum lines, can be very readily controlled.

In an advantageous embodiment, the hollow-cylindrical portion has an oval cross section. Here, the cross section is seen parallel to the rail or to a plane which is defined by end edges of the bearing legs. As a result, the orientation of the film situated thereon is controlled since it preferably folds along the longer extent of the hollow-cylindrical portion. Consequently, excessive wrinkling can be avoided and, upon evacuation, the film adapts harmonically to the surface of the connection adapter.

In an advantageous embodiment, at least one dimension of an extent of the cross section tapers up to an upper side in a direction facing away from the bearing side. The film also hugs the hollow-cylindrical portion particularly smoothly along lateral sides thereof. The cross section is determined as mentioned above. In this sense, a dimension can be the width or the depth of the cross section. An upper side of the hollow-cylindrical portion therefore has the smallest cross section. Here, the film can extend upwardly in a smooth manner from a bearing region on the legs over the hollow-cylindrical portion, and wrinkling or the like on the lateral surfaces is largely avoided by virtue of the slight conical shape.

In a particularly preferred embodiment, a shoulder is arranged in the connection opening in order to delimit a plug-in depth of an external fluid line. A user can therefore plug the external fluid line into the connection opening until a shoulder discernibly prevents further pushing. It can thus not only be guaranteed that a necessary plug-in depth is reached but also that damage to the fiber semi-finished product is prevented.

In an advantageous embodiment, the legs of the connection adapter are configured as planar bodies which have a substantially constant length. The connection adapter can thus hug the inner fluid line in a very flush manner, said fluid line, in particular as a resin line, often having a roughly triangular, largely constant outer contour.

The connection adapter is preferably produced from a plastic. Here, care should be taken to ensure that the material has a sufficient temperature resistance. The curing of the components for which the vacuum infusion method is used can be carried out at a temperature from 140 to 180 degrees. Since, after evacuation, the connection adapter remains on the mold and underneath the vacuum film until the curing process has finished, it should therefore have a corresponding temperature resistance.

The connection adapter is particularly preferably produced from an at least rubber-like material. The vacuum film produces a certain contact pressure on the connection adapter, with the result that the latter presses onto the inner fluid line in order to seal the bore, or the cutout, situated therein on the edge. A rubber-like material can be considerably better suited for this task than a rigid and dimensionally stable material.

The connection adapter can be produced by a molding method. When using a rubber-like material, a moldable material based on a two-component system can be realized. As mentioned above, care should be taken to ensure that the material is temperature-stable at a temperature of at least 180° C. It would be advantageous to select a material which is temperature-stable at a temperature of at least 200° C.

The invention further relates to a system for producing a component from a fiber composite material, having a mold with a bearing surface, at least one inner fluid line, at least one connection adapter for each inner fluid line, and a vacuum film for covering a semi-finished product, and a vacuum pump.

Of course, further components are conceivable which are necessary for the optimized implementation of a method carried out with the system.

Here, at least one of the inner fluid lines can be an inner resin line. Alternatively or additionally to this, at least one of the inner fluid lines can be an inner vacuum line.

In an advantageous embodiment, the inner fluid line has an omega-shaped profile cross section. Such a configuration of a fluid line is very widespread particularly in the case of resin lines and can here positively influence the distribution of the inflowing resin.

### BRIEF DESCRIPTION OF THE DRAWINGS

Further features, advantages and application possibilities of the present invention emerge from the following description of the exemplary embodiments and the figures. In these, all of the features described and/or graphically presented form by themselves and in any desired combination the subject matter of the invention, even independently of how they are grouped together in the individual claims or the way in which the claims refer back to one another. Furthermore, in the figures the same reference signs stand for objects that are the same or similar

FIG. 1 shows an inner resin line with a connection adapter arranged thereon.

FIG. 2 shows an inner resin line with a bore for a fluid connection to a connection adapter.

FIG. 3 shows a connection adapter in an oblique view from above.

FIG. 4 shows a connection adapter from the side.

FIG. 5 shows a system for producing a component from a fiber composite material in a schematic illustration.

### DETAILED DESCRIPTION OF THE PREFERRED EMBODIMENTS

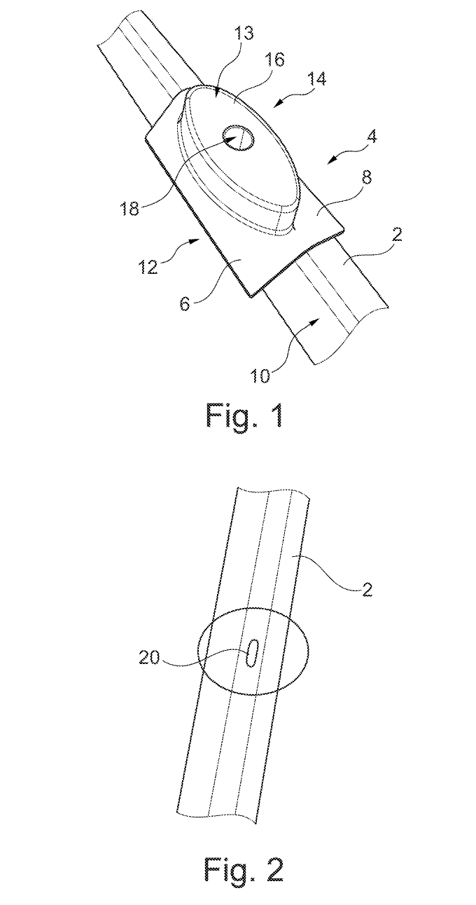

FIG. 1 shows an inner fluid line 2, which is, by way of example, an inner resin line for channeling resin, for a mold, and a connection adapter 4 placed thereon. The latter has two bearing legs 6 and 8 which are configured substantially as planar elements and are arranged flush on a surface 10 of the inner fluid line 2. The connection adapter can therefore produce a good, flush contact with the inner fluid line 2.

The material of the connection adapter 4 should particularly have sufficient temperature resistance and have optimum shape elasticity. It is appropriate for this purpose to use a moldable, elastic and preferably rubber-elastic material which tolerates temperatures of above 180° C. It may be appropriate to use a moldable high-temperature rubber which crosslinks at room temperature, for example. Such materials are available with a use temperature of considerably above 200° C. In addition, the fluid connection between the inner fluid line 2 and the connection adapter 4 can be improved by a rubber-like material since, upon evacuation, a film presses the connection adapter 4 onto the line 2 and at the same time provides sealing along the contact surfaces.

In addition to a bearing side 12 which contains the two bearing legs 6 and 8, the connection adapter 4 also has a connection side 14 which has a hollow-cylindrical portion 16. This is equipped, by way of example, with an oval or elliptical cross section.

From a cut edge between the bearing legs 6 and 8 and the hollow-cylindrical portion 16, the latter extends in a direction facing away from the bearing side 12. Here, the cross section tapers up to an upper side 13 in a slightly conical shape, for example.

The hollow-cylindrical portion 16 has a connection opening 18 which extends completely through the hollow-cylindrical portion 16 and down through the bearing legs 6 and 8 and opens into a connecting opening 11. The connection opening 18 serves for receiving a suitable external fluid line through which, in this example, resin is channeled into the inner fluid line 2. For this purpose, as shown further below in FIG. 2, the inner fluid line 2 is provided with a cutout 20. This can be produced separately in an inner fluid line 2 as required, or the inner fluid line 2 can already be provided during its production with a plurality of cutouts 20 arranged at suitable points.

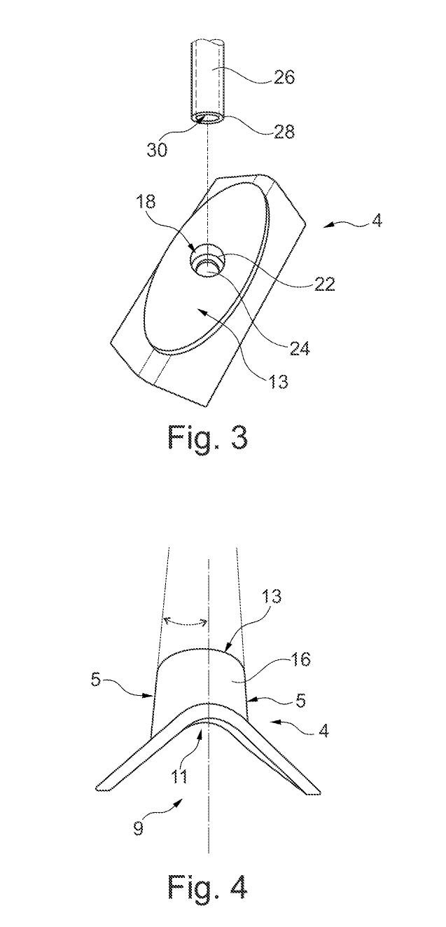

FIG. 3 shows the connection adapter 4 from above in an isolated, three-dimensional illustration. A detail of the connection opening 18 which extends into the interior of the connection adapter 4 is visible here. In the interior of the connection opening 18 there is arranged, at a predetermined spacing from the upper side of the hollow-cylindrical portion 16, a shoulder 22 which has a shoulder opening 24 which has a smaller diameter than the connection opening 18. As a result, an external fluid line 26, which is configured, for instance, as a hose, can be plugged into the connection opening 18. Upon being plugged in, an annular end surface 28 of the external fluid line 26 comes into surface contact with the shoulder 22. Further pushing of the fluid line 26 is then no longer possible. At the same time, a fluid connection is produced between the hose 26 and the shoulder opening 24 through an opening 30 of the external fluid line 26 and the shoulder opening 24, with the result that, when the connection adapter 4 is placed, a fluid connection with the inner fluid line 2 is finally produced.

FIG. 4 shows a side view of the connection adapter 4. Particularly visible here is the slight conical shape of the hollow-cylindrical portion 16. For example, a cone angle is provided such that lateral surfaces 5 of the hollow-cylindrical portion 16 taper with respect to the vertical or a direction of extent of the openings 11 and 18 by an angle in a range of somewhat less than 1° to 25°. Of course, other cone angles are also possible.

The angle between the two bearing legs 6 and 8, which enclose an interspace 9 widening in a direction facing away from the connection side 14, could be roughly 90°. However, this is dependent on the configuration of the inner fluid line 2 used. If a rubber-elastic material is used for the production of the connection adapter 4, very precise adaptation of the shape of the connection adapter 4 to the inner fluid line 2 is not necessary. However, a connecting opening 11 which is in fluid communication with the connection opening 18 is situated at a cut edge between the two bearing legs 6 and 8. As a result, resin introduced into the connection opening 18 can pass into the cutout 20 of a rail 2.

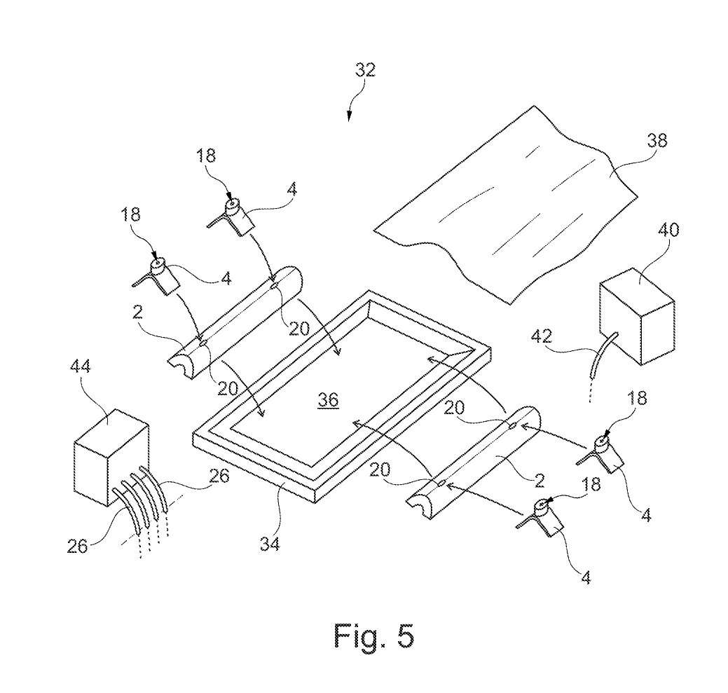

Finally, FIG. 5 schematically shows a system 32 for producing a component from a fiber composite material. A mold 34 serves for receiving fiber semi-finished products on a bearing surface 36 which determines the shape of the component. To carry out a resin infusion there is illustrated, for example, an inner resin line 2 which can be laid on the fiber semi-finished product. In addition, a vacuum line 42 is provided by means of which a vacuum can be applied which leads to the resin infusion. For preparation there are provided, for example, two cutouts 20 on the inner resin line 2 on each of which a connection adapter can be placed. Arranged on the vacuum line 42 are cutouts on which connection adapters 4 are likewise placed. For vacuum-tight covering there is further provided a vacuum film 38 which is laid on the fiber semi-finished product and the inner fluid line 2 situated thereon. Here, for the sake of simplicity, strip-type seals and other devices for assisting the vacuum infusion are not illustrated. To produce the negative pressure there is provided a vacuum pump 40 which can be brought into fluid communication via a line 42 with the mold 34 or the bearing surface 36 via the cutouts and the connection adapter 4.

The connection adapters 4 according to the invention allow the introduction of resin at a plurality of points of an inner resin line 2 without an additional hose having to be laid underneath the vacuum film 38 that presses on a component to be produced and could limit its shape accuracy. In addition, the volumetric flow of resin is adjustable by choosing the suitable number of connection adapters 4. All the connection adapters 4 can be connected to in each case an external resin line 26, which resin lines could, by way of example, be led out from a resin container 44 as separate lines or can each be realized as a branch of a larger resin line. This analogously applies to the connection adapters 4 which can be fitted to the inner vacuum line 42.

Furthermore, it should be pointed out that features which have been described with reference to one of the above exemplary embodiments can also be used in combination with other features of other above-described exemplary embodiments.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise,” “having” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A connection adapter to connect an inner fluid line of a device for vacuum infusion, comprising;
a bearing side and a connection side,
the bearing side having two bearing legs for placing on a fluid line which enclose an interspace which widens outwardly in a direction facing away from the connection side,
the connection side having a hollow-cylindrical portion with a connection opening facing away from the bearing side, with a connecting opening projecting into the bearing side, and with a fluid connection between the connection opening and the connecting opening.

2. The connection adapter according to claim 1, wherein the hollow-cylindrical portion has an oval cross section.

3. The connection adapter according to claim 1, wherein at least one dimension of an extent of a cross section tapers up to an upper side in a direction facing away from the bearing side.

4. The connection adapter according to claim 1, wherein a shoulder is arranged in the connection opening in order to delimit a plug-in depth of an external fluid line.

5. The connection adapter according to claim 1, wherein the bearing legs of the connection adapter are configured as planar bodies which have a substantially constant length.

6. The connection adapter according to claim 1, wherein the connection adapter is produced from a plastic.

7. The connection adapter according to claim 1, wherein the connection adapter is produced from an at least rubber-like material.

8. A system for producing a component from a fiber composite material, having a mold with a bearing surface, inner fluid lines, at least one connection adapter for each inner fluid line, a vacuum film for covering a semi-finished product, and a vacuum pump.

9. The system according to claim 8, wherein at least one of the inner fluid lines is an inner resin line.

10. The system according to claim 8, wherein at least one of the inner fluid lines is an inner vacuum line.

11. The system according to claim 8, wherein the inner fluid line has an omega-shaped profile cross section.
